(19) United States
(12) Patent Application Publication
(10) Pub. No.: US 2004/0023912 A1
(43) Pub. Date: Feb. 5, 2004

(54) Method of diagnosis and treatment of breast lesions

(75) Inventor: David Hung, Redwood City, CA (US)

Correspondence Address: BANNER & WITCOFF, 1001 G STREET N W, SUITE 1100, WASHINGTON, DC, 20001, US

(73) Assignee: Cytyc Health Corporation, Boxborough, MA

(21) Appl. No.: 10/388,764

(22) Filed: Mar. 17, 2003

Related U.S. Application Data
(60) Provisional application No. 60/364,136, filed on Mar. 15, 2002.

(51) Int. Cl. A61K 49/00; A61K 48/00; A61K 31/522

(52) U.S. Cl. 514/44; 424/9.1; 514/263.31

(57) ABSTRACT

The present invention relates to an apparatus and method for administering diagnostic and therapeutic agents into a breast duct. A thymidine kinase vector may be administered into a breast duct in conjunction with an acycloguanidine compound for enhanced imaging on PET scanning of the breast to detect breast lesions. The thymidine kinase phosphoraylates the acycloguanidine compound to enhance visibility of lesions on PET scan. An acyclic nucleoside compound may be administered into the breast duct to form phosphorylated adducts that phosphorylate DNA and kill thymidine kinase expressing cancer cells.

### RELATED APPLICATIONS

This application claims benefit under 37 CFR §1.78 of provisional application Serial No. 60/364,136, filed Mar. 15, 2002. The full disclosure of the application is incorporated herein by reference.

### TECHNICAL FIELD

The present invention relates to a method and apparatus for management of breast lesions and in particular enhancing imaging of breast tumors.

### BACKGROUND OF THE INVENTION

Breast cancer is a major cause of death in women. It is estimated that up to 10% of women in the United States are at risk of developing breast cancer in their lifetime. Methods of early detection have been developed such as physical examinations, regular self-examinations, mammography or tissue biopsy, however, inherent features of these methods limit their utility. Physical examinations and self-examinations may depend on the skill of the examiner and some lesions, particularly small-sized lesions, may be overlooked. Mammograms may sometimes be difficult to interpret in more dense breast tissue. Furthermore, mammograms may lack optimal sensitivity such that breast lesions may be present for many years and may develop to an advanced stage of disease before they are detectable on mammogram. Because advanced stage disease often carries a poor prognosis, reliance on mammogram may be less than optimal.

Tissue biopsy often requires a palpable lesion before sampling may be performed effectively at which time the lesion may have progressed to an advanced stage and may carry a poorer prognosis. If the lesion is more advanced, there may be a higher risk of treatment failure. This problem may be offset if the lesion could be detected earlier. Tissue biopsies may also cause tissue artifacts that may be problematic for proper diagnosis. For example, fine needle aspiration (FNA) may cause local reactions at the site of the biopsy such as scarring or inflammation that may make it difficult to visualize the tumor both grossly or microscopically. In such a situation, proper diagnosis is hampered and the tumor may be missed. Also, tissue biopsies may even cause spread of the tumor if the tumor or tumor cells are dislodged from the main tumor site into surrounding tissues. In addition, tissue biopsy is an invasive procedure that may cause patient discomfort and inconvenience.

Diagnosis of breast lesions has been accomplished through nuclear medicine imaging techniques. Positron Emission Tomography (PET) scanning, a type of nuclear medicine imaging, has proven to be useful in detection of tumor masses, differentiating between benign and malignant tumors, demonstrating spread of malignant tumors or monitoring course of therapy. PET scanning has been employed in the detection of a variety of tumors including breast, colon, prostate, lung and other organs. In the breast, imaging with conventional modalities such as X-rays, CT scans, MRIs or mammograms often does not adequately reveal underlying tumor pathology where PET scanning may prove more effective.

In PET scanning, a positron (i.e., “anti-electron”) collides with an electron causing annihilation and emission of gamma rays in opposite directions, which are recorded by a detection device and converted into an image of the organ of interest. Thus, low levels of radiation emitted from a patient of gamma rays following administration to the patient of a radioactive pharmaceutical provides desired images. The administered radioactive pharmaceutical may be any number of compounds or may resemble a naturally occurring compound, such as glucose (e.g., fluoro-deoxy-glucose, FDG), 6-F-Dopa, radioactive water, or 1-(3-[¹⁸F] fluoro-2-hydroxypropyl)-2-nitroimidazole (¹⁸FMISO), for example. It has also been shown that acyclic nucleotide compounds such as acycloguanidines may also be effectively utilized in PET scanning.

Following PET scanning of breast lesions, a tumor mass may be identified at which time proper therapy may be initiated. For example, if a malignant breast tumor is detected through PET scanning, chemotherapy may then commence. However, there is currently no method of utilizing PET scanning in the direct therapy of breast tumors.

Therapeutic uses of Thymidine Kinases (TK) are described. Thymidine kinases (TK) have been used to induce cell death in cells that express TK thus providing a means for therapy of cancer such as breast cancer. Tumor cells that are transfected with TK genes (e.g., Herpes simplex virus thymidine kinase, HSV-tk) have been demonstrated to be sensitized to acyclic nucleotide compounds such as Gancyclovir (GCV), an acyclic nucleotide analogue of 2′-deoxyguanosine. It is most likely that GCV is phosphorylated first to the monophosphate form, then to the di- and triphosphate forms by cellular kinases, then passively transferred to adjacent cells through cell-to-cell connections. The phosphorylated adducts thus formed act to phosphorylate DNA and kill the cells. GCV triphosphate may inhibit DNA synthesis by competitive inhibition of DNA polymerases or incorporation into DNA and termination of DNA elongation. Methods of killing breast cancer cells in a tumor involving TK are described in U.S. Pat. No. 6,096,718, Weitzman et al., issued Aug. 1, 2000, WO 97/05898, Sukumar, published Feb. 20, 1997, and Sukumar, U.S. Pat. No. 5,763,415, issued Jun. 9, 1998. Both of these references are incorporated herein in their entirety by reference. However, more effective therapy of cancer, such as breast cancer, may necessitate a means of diagnosing and detecting as well as killing cancer cells.

Thus, there exists a need for a method of effectively performing and enhancing nuclear imaging of breast tumors via administering imaging agents while directly treating suspicious lesions and limiting radiation exposure to the patient.

### SUMMARY OF THE INVENTION

The present invention relates to a method for imaging breast lesions in a breast wherein a vector comprising a thymidine kinase gene is delivered into a breast duct of the breast, an acycloguanidine compound is administered into the breast duct and the breast is imaged. The thymidine kinase gene may be a herpes virus thymidine kinase gene which may be administered either simultaneously or sequentially with the acycloguanidine. The invention further relates to administering an acyclic nucleotide compound such as gancyclovir into a breast duct to treat breast lesions.

Although PET scanning has been performed for cancer diagnosis, there has been no method for effectively performing imaging of the breast by local administration of desired agents into a breast duct system, including one or more ducts, and subsequent therapy of detected breast tumors by local administration of therapeutic agents into the breast duct system. In the present invention, it was discovered that more effective dosing may be achieved utilizing lower doses of agents by local administration of agents directly into the breast duct system. Moreover, harmful side effects of the agents of interest can be minimized by the local administration as opposed to systemic administration of the agents. Examples of side effects from systemic administration of such agents that can be minimized by local administration of the agents directly into the breast duct system include nausea, vomiting, diarrhea, neutropenia, etc.

Further, by combining diagnosis of breast tumors using PET scanning with treatment of breast cancer using agents that are activated or phosphorylated in the presence of thymidine kinase, more effective and efficient management of breast cancer can be achieved by diminishing the number of steps and the complexity of first diagnosing breast cancer and then treating breast cancer. By simplifying the process, diagnosing and treating breast cancer is not only simplified but is also expedited thus leading to earlier therapy of potentially malignant breast lesions.

### BRIEF DESCRIPTION OF THE DRAWINGS

FIG. 1 illustrates an exemplary apparatus for administering compounds into breast tissue.

FIG. 2 illustrates an exemplary apparatus for administering compounds into a breast duct system involving a single port.

FIG. 3 illustrates an exemplary apparatus for administering compounds into a breast duct system involving a Y-Tube-Shaped catheter.

### DETAILED DESCRIPTION OF THE INVENTION

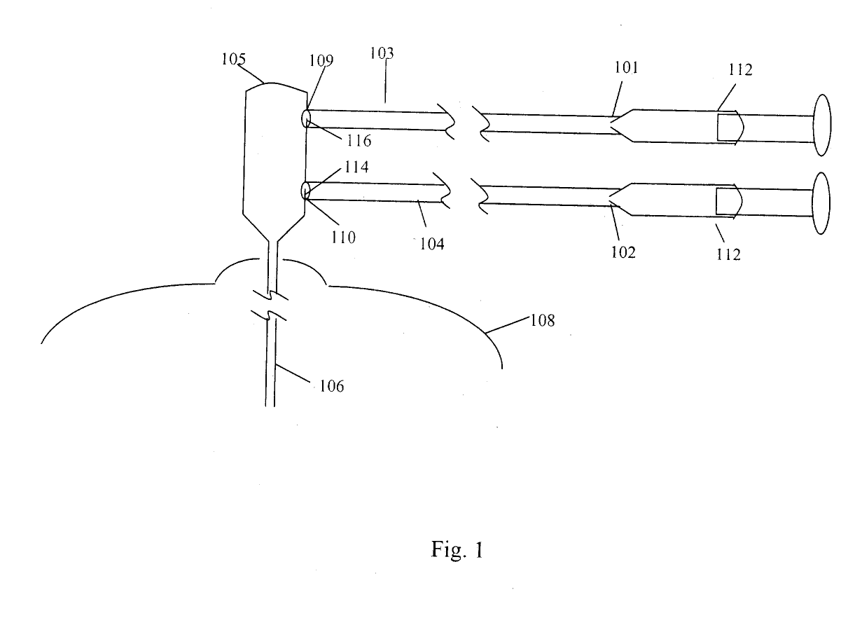

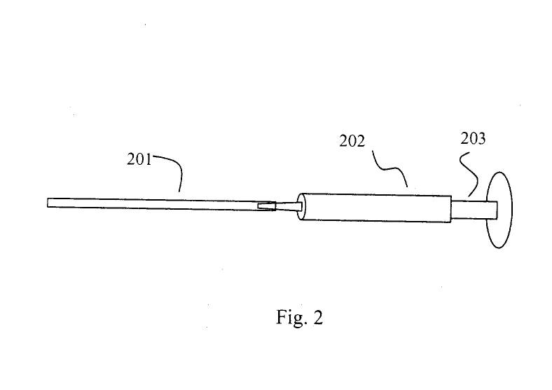

The present invention provides a method and apparatus for administering diagnostic or therapeutic agents such as agents in the management of breast lesions. FIG. 1 illustrates an exemplary embodiment of an apparatus for administering diagnostic or therapeutic agents for management of breast lesions in which multiple ports are utilized to introduce material into a breast duct. FIG. 2 illustrates an alternative embodiment of a device for administering agents in which a single port is utilized to introduce material into a breast duct. It should be noted that the illustrated devices are for illustration purposes only and are not meant to limit the present invention as many similar devices may be utilized by a skilled artisan without departing from the scope or spirit of the invention.

The apparatus of the present invention may be introduced into a breast duct through a ductal opening in the nipple and diagnostic or therapeutic agents may be locally introduced into the breast duct. Thus, without the need for percutaneous introduction of material, breast lesions may be identified early in their formation even before they are grossly visible or palpable. As disclosed in U.S. Pat. No. 6,096,718, Weitzman et al., issued Aug. 1, 2000 and incorporated herein in its entirety by reference, thymidine kinase genes kill tumor cells when expressed in the presence of certain agents such as gancyclovir, which is an acyclic nucleoside analogue of 2′-deoxyguanosine. Hence, thymidine kinase vectors may be administered in conjunction with gancyclovir which has been approved for humans and are in clinical trials. When the thymidine kinase vectors are administered in conjunction with gancyclovir, the agents may be administered either simultaneously or sequentially in either order and separated by a time period that is sufficiently short such that the first administered agent remains effective at the time of administration of the second agent. For example, the thymidine kinase vector may be administered into a breast duct at tₒ. The gancyclovir may be introduced into the breast duct at time (tₒ+tₓ), where tₓ is an amount of time between administration of the thymidine kinase vector and the gancyclovir. The time between administrations, tₓ, may vary but is chosen such that the agents remain effective at the time of administrations. After a predetermined amount of time following the administration of the thymidine kinase gene, transfection of the gene may occur. Infusion of gancyclovir may range from 12-96 hours following the adminstration of the thymidine kinase, for example. Longer wait times may also be possible as cells may remain transfected for greater than 96 hours after administration of the vector. For example, lag times between the administration of the thymidine kinase gene and the administration of the gancyclovir may range up to 10-25 days or higher. Likewise, lag times between the administrations may be less than 12 hours, however, transfection may not be optimal if the time waited is too short such that there is inadequate transfection.

Gancyclovir may be administered prior to the thymidine kinase vector. In this embodiment, the gancyclovir may be administered optimally up to 15-30 hours prior to the administration of the thymidine kinase vector. Gancyclovir may also be administered greater than 30 hours prior to the administration of the thymidine kinase vector up to the point where the gancyclovir is cleared which is based on the half-life of gancyclovir. Likewise, gancyclovir may be administered less than 15 hours prior to the adaministration of the thymidine kinase vector. This would permit treatment of tumor cells in the breast duct with gancyclovir after transfection of cells with the thymidine kinase vector. It should be noted that there is a wide range of variation of administration of agents into the breast duct as described. The embodiments described herein are merely illustrative and are not intended to limit the present invention.

Administration of the gancyclovir and the thymidine kinase vector may occur in a single duct or may be performed on a plurality of ducts. For example, a catheter may be inserted into a single breast duct and the thymidine kinase vector may be administered into the single breast duct. Imaging, such as PET imaging, may be performed for localization of any tumor present to a particular duct or duct system. Alternatively, the gancyclovir and the thymidine kinase may be administered into a plurality of breast ducts such that a plurality of breast ducts receive the agents and imaging of the plurality of ducts may be accomplished to diagnose or treat any tumor present.

Thymidine kinases also phosphorylate a variety of acycloguanidines. Acycloguanidines may be used in positron emission tomography (PET) scanning for visualization of tumor masses. However, use of acycloguanidines in PET scanning may not be effective unless the acycloguanidines are in a phosphorylated state as they may not be visible or are inadequately visible if not phosphorylated. Phosphorylation of acycloguanidines may thus provide for enhanced visibility of tumor masses in breast cancer.

As exemplified in FIG. 1, which demonstrates an illustrative embodiment of a ductal access device 100 of the present invention, the exemplary apparatus for introducing material into a breast duct or obtaining a biological sample from a breast duct contains an access device such as a catheter or cannula that may be inserted into the breast duct. A biological sample may be obtained from the breast duct system via this access device or a separate catheter or cannula. U.S. patent application Ser. No. 09/473,510, David Hung, et al., filed Dec. 28, 1999, which is incorporated herein in its entirety, discloses an exemplary apparatus having an elongated ductal access device that can be used with the present invention for positioning within a breast duct.

FIG. 1 illustrates one exemplary embodiment of the ductal access device 100 of the present invention. The ductal access device 100 contains a hollow elongated member with an internal lumen which can include a catheter or a cannula having an internal lumen extending between its ends (e.g., a catheter 106). The catheter 106 may be for positioning within a breast duct and a main chamber or manifold 105 in fluid communication with the catheter 106. The main chamber 105 has an internal volume and an internal diameter that is greater than that of the catheter 106. The main chamber 105 also includes a first port 110 and a second port 109. These ports 109, 110 can be placed at any position discussed in U.S. patent application Ser. No. 09/473,510. For example, the second port 109 can be placed at the terminal end of the main chamber 105 and inline with the catheter 106. Additionally, the first port 110 can be positioned as close to the catheter 106 as possible. Moreover, these ports 109, 110 can be vertically aligned with each other along the wall of the main chamber 105 or offset around the circumference of the main chamber 105.

Fluids and other materials can be introduced into and removed from the main chamber 105 through either of the illustrated ports 109, 110. As illustrated in FIG. 1, the first port 110 is connected to a first conduit 104 that has a port 102 for receiving an instrument such as a syringe 112. The second port 109 is connected to a second conduit 103 that has a port 101 for receiving a syringe 112. The syringes 112 can be replaced by any known collection and/or infusion device.

As discussed above, the port 102 and conduit 104 can be used to infuse material into the main chamber 105 and into the duct via the catheter 106. The material or fluid is placed into the first port 102 and positive pressure is exerted at the first port 102 to expel the material or fluid into the main chamber 105 and into the breast duct system via the catheter 106. Alternatively, the material or fluid to be introduced into the breast duct system may be placed into the second port 101 and expelled into the main chamber 105 through the second conduit 103 by exerting positive pressure at the second port 101. The material or fluid may thus be administered into the chosen breast duct.

As previously mentioned, the port 101 and the conduit 103 can be used to introduce material into the breast duct system or collect material received from the duct (duct system) and contained in the main chamber 105. For example, negative pressure may be exerted at the first port 109 by the operation of the syringe 112 connected to the port 101. This action produces a negative pressure in the main chamber 105 and draws the material obtained from the breast duct and residing in the main chamber 105 into the conduit 103 and the syringe 112 or other collection device.

The above descriptions of the ports and conduits used to introduce fluid into the duct and collect material from within the duct are merely exemplary. Either set of conduits and ports can be used to perform either of these functions. Extraction of biological material which can include ductal fluids, cells (cell clumps) and the ductal wash fluid from the breast duct system may be accomplished by externally massaging the breast after the ductal wash fluid has been introduced into the duct. Additionally, negative pressure within the main chamber 105 can be created by the operation of one or more of these syringes 112.

As shown in FIG. 1, valves 114, 116 may regulate the flow of material or fluid into and out of the main chamber 105 through the input port 110 and output port 109, respectively. The catheter 106 may have an internal lumen of a diameter sufficiently sized such that insertion into a breast duct system is facilitated while permitting the passage of desired agents and material. The catheter 106 may, for example, have a lumen diameter of 0.007 inches (or 0.178 mm) or greater, or a lumen diameter in the range from 0.007 inches (or 0.178 mm) to 0.047 inches (or 1.19 mm). Further, the catheter 106 may contain indicia on its surface to indicate the depth of insertion such that a user may be fully aware of the depth of insertion of the catheter 106 during insertion of the catheter 106 into the breast duct. Further, the catheter 106 may contain a safety mechanism such as a stop element such that the catheter 106 may not be further advanced into the breast duct system after a certain depth is attained. Such a stop element may be variously designed but may comprise, for example, a collar affixed to or formed on an exterior surface of the catheter 106, the collar being of a width greater than the diameter of the catheter 106.

FIG. 2 illustrates another exemplary embodiment of an apparatus for administering agents into a breast duct system. In this embodiment, the apparatus is a single lumen device comprising a catheter 201 in connection with a syringe 202. The syringe 202 enables introduction of desired agents into the breast duct system. A plunger 203 may be situated at a top end of the syringe 202, for example, and may be used to introduce agents contained within the syringe 202 into the breast duct system (not shown). Different syringes can be used sequentially or one syringe can include all elements to be introduced into the breast duct system.

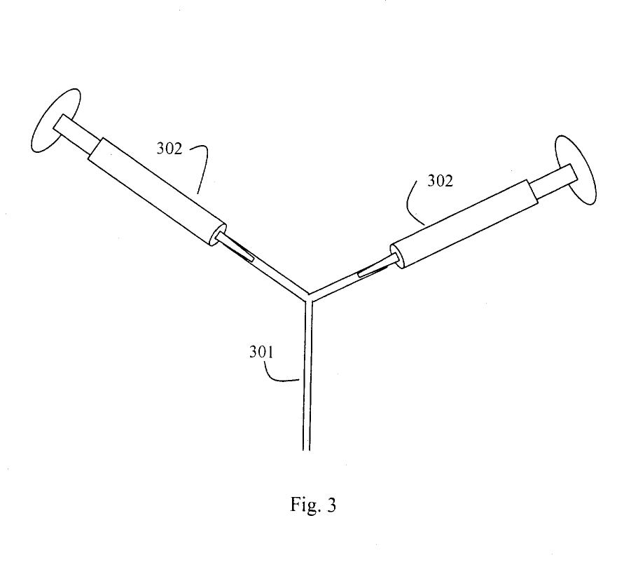

FIG. 3 illustrates another exemplary embodiment of an apparatus for administering agents into a breast duct system. In this embodiment, a syringe is connected to a Y-tube-shaped catheter at each of a plurality of proximal ends of which two are shown. The distal end of the Y-tube-shaped catheter may be inserted into a breast duct system via a nipple surface. Desired agents may be introduced into the breast duct system from any of the plurality of proximal ends of the Y-tube-shaped catheter. This exemplary embodiment allows multiple agents to be administered separately or allows the mixing of separately administered agents for simultaneous administration.

It will be appreciated that the disclosed exemplary embodiments of an apparatus for administering agents into a breast duct system are for illustration purposes only and are not intended to limit the present invention. Any suitable device suitable for injecting or infusing fluid into a duct may be utilized for introducing desired agents into a breast duct system may be employed without deviating from the scope or spirit of the present invention.

In an exemplary embodiment of the present invention, administration of agents may be accomplished in the breast duct to thereby reduce the risk of side effects and to enhance the anti-tumor effect of breast tumors while performing imaging of the breast tumor. A thymidine kinase gene such as herpes virus thymidine kinase (HSV-tk) gene may be administered into a breast duct in conjunction with an acycloguanidine for PET scan imaging. For example, thymidine kinase phosphorylates the acycloguanidine causing the acycloguanidine to become more effectively visible on PET scanning. The thymidine kinase (HSV-tk) gene in this example may be administered simultaneously with the acycloguanidine or may be administered sequentially with the administration of the acycloguanidine. For example, in one illustrative embodiment, the thymidine kinase may be administered followed by the administration of the acycloguanidine wherein the acycloguanidine is administered 12-96 hours after the administration of the thymidine kinase gene. The administration of the acycloguanidine may also be administered greater than 96 hours after the administration of the thymidine kinase gene if the phosphorylation of the acycloguanidine may be effected. Optimally, the administration of the acycloguanidine may be up to 15 days or longer after the administration of the administration of the thymidine kinase gene. Alternatively, the thymidine kinase gene may be administered after the administration of the acycloguanidine. In this exemplary embodiment, the administration of the thymidine kinase gene may be administered 15-30 hours after the administration of the acycloguanidine. This would permit the phosphorylation of the acycloguanidine by the thymidine kinase such that the acycloguanidine may be more effectively visualized on PET scanning. The administration of the thymidine kinase gene may also occur greater than 30 hours after the administration of the acycloguanidine or less than the 15 hours after the administration of the acycloguanidine such that the visualization of any tumor is more effectively accomplished on PET scanning, for example. It should be noted that there is a wide range of variation of administration of agents into the breast duct as described. The embodiments described herein are merely illustrative and are not intended to limit the present invention. Therefore, when the thymidine kinase genes are administered in conjunction with an acycloguanidine for PET scan imaging as described, the agents may be administered either simultaneously or sequentially in either order and separated by a time period that is sufficiently short such that the first administered agent remains effective at the time of administration of the second agent. In this example, HSV-tk and the acycloguanidine may be administered into a breast duct via the apparatus illustrated in FIG. 1 or any device suitable for injecting or infusing fluid into a duct. The catheter 106 may be introduced into the breast 108 via a breast duct and an effective dose of the vector (not shown) may be introduced through the input device 102 and input port 104 into the main chamber 105 and into the breast 8 via the catheter 106. Alternatively, the effective dose of the vector may be introduced into the main chamber 105 through the alternate output port 103. The effective dose of the vector may be determined during the course of therapy and may depend on a variety of factors such as, for example, the size or extent of the breast duct system or variation in breast tissue in terms of size or resistance or complexity of the breast duct system. A skilled artisan may determine a proper amount for administration by, for example, slowly introducing material until resistance is detected. Slow administration will not only reduce the risk of duct rupture but also ensure that substantially all of the branches in the breast duct system receives the agent. A test may be first performed by introducing saline. This may provide information on the volume of agent that may be introduced without rupture. For example, up to 17 ml may be slowly administered into a breast duct system over 5 minutes during any testing procedure or other known procedure for placing fluid(s) in a duct such as ductal lavage.

The vector may be administered in conjunction with the acycloguanidine. When the vectors are administered in conjunction with the acycloguanidine, the agents may be administered either simultaneously or sequentially in either order and separated by a time period that is sufficiently short such that the first administered agent remains effective at the time of administration of the second agent. For example, the acycloguanidine may be introduced from a first port 102, through the first conduit 104 and into the breast 108 through the main chamber 105 and catheter 106. Alternatively, the acycloguanidine may also be introduced into the main chamber 105 through the second port 101 and the second conduit 103. The vector (not shown) may be introduced into the breast duct simultaneously with the acycloguanidine. For example, the vector may be introduced via the first conduit 104 into the breast duct simultaneously with introduction of the acycloguanidine via the second conduit 103.

Alternatively, the acycloguanidine may be administered sequentially with the vector. For example, a syringe 202 connected to a catheter 201 may contain the acycloguanidine. The catheter 201 may be introduced into a breast duct system via a breast nipple surface. A plunger 203 on one end of the syringe 202 may be activated to cause the acycloguanidine within the syringe 202 to enter into the breast duct system via the catheter 201. Subsequently, the vector may be placed into the syringe 202 and the plunger 203 may be activated such that the vector within the syringe 202 enters the breast duct system via the catheter 201. Alternatively, they can be administered at the same time using the syringe 202.

Also, two syringes may be employed, each syringe being connected to a Y-tube-shaped catheter. A distal end of the Y-tube-shaped catheter 301 may be introduced into a breast duct system via a breast nipple surface. One syringe 302 may contain the acycloguanidine and another syringe 302 may contain the vector. The acycloguanidine may be introduced into the Y-tube-shaped catheter by, for example, applying pressure at the plunger of the syringe 302 containing the acycloguanidine. The acycloguanidine is thus passed into the Y-tube-shaped catheter 301 and into the breast duct system. Subsequently, the vector may be introduced in a similar fashion into the Y-tube-shaped catheter and directed into the breast duct system. The vector may be introduced via any of the syringes 302. Alternatively, the acycloguanidine and the vector may be administered simultaneously into the Y-tube-shaped catheter 301 via one or both syringes such that the acycloguanidine and the vector may be permitted to mix within the Y-tube-shaped catheter 301 prior to entering the breast duct system.

It will be appreciated that any number of devices may be used to introduce the acycloguanidine and vector into the breast duct system in any order or simultaneously without deviating from the spirit and scope of the present invention. For example, known substitutes for syringes can be used.

A breast tumor may be effectively identified through PET scanning as described above. PET scanning provides information on the malignant or benign nature of the breast tumor, the location of the tumor, response to therapy, etc. In the method of the present invention, acycloguanidine may be administered into a breast duct system in a breast, suspected of having a breast lesion such as breast cancer. Acycloguanidine may be used as an agent in PET scanning to detect the presence of breast tumor, however, acycloguanidine can more optimally be used in PET scanning to detect breast tumor if phosphorylated. Therefore, a thymidine kinase gene may be introduced into the breast duct system in conjunction with the acycloguanidine. When the thymidine kinase gene is administered in conjunction with acycloguanidine, the agents may be administered either simultaneously or sequentially in either order separated by a time period that is sufficiently short such that the first administered agent remains effective at the time of administration of the second agent. The combination of the thymidine kinase gene and the acyloguanidine results in phosphorylation of the acycloguanidine. The acycloguanidine is thus “activated” to be useful in detection of breast tumor in PET scanning.

If a breast tumor is detected on PET scanning after addition of acycloguanidine into the breast duct system and phosphorylation of the acycloguanidine with the thymidine kinase gene, the detected breast tumor may subsequently be treated. In the method of the present invention, after detection and qualification of the tumor with TK and acycloguanidine, therapy of the breast tumor may be affected by administering gancyclovir into the breast duct. TK, having been previously administered during PET scanning serves an additional role when in the presence of gancyclovir. Gancyclovir administered in conjunction with TK results in phosphorylated adducts that phosphorylate DNA of the tumor cells and kill the tumor cells. For example, gancyclovir may be phosphorylated and inhibit DNA synthesis by competitive inhibition of DNA polymerases or incorporation into DNA and eventual termination of DNA elongation. The thymidine kinase gene having been transduced into the breast duct with acycloguanidine for PET scanning, the TK expressing tumor cells are killed by gancyclovir.

For example, acycloguanidine may be administered into a breast duct system in a breast suspected of containing a breast tumor via a first port 102 and first conduit 104. The acycloguanidine may pass into a main chamber 105 and into the breast duct system via a catheter 106. Optimal use of acycloguanidine in PET scanning of breast tumors my be accomplished by phosphorylation of the acycloguanidine with thymidine kinase which may be administered into the breast duct system either simultaneously or sequentially with the acycloguanidine. For example, the thymidine kinase may be administered via a second port 101 and second conduit 103 and into the breast duct system through a main chamber 105 and catheter 106. The acycloguanidine becomes phosphorylated in the presence of TK and more effective PET scanning of the breast tissue is performed.

If a tumor is detected on PET scanning, therapy of the detected tumor mass may be performed utilizing the TK previously transduced in the breast duct system. In this embodiment, Gancyclovir, or an effective amount of an acyclic nucleoside compound, may be administered into the breast duct system via a first port 102 or a second port 101. In this embodiment, Gancyclovir may be introduced into the breast duct system via a main chamber 105 and catheter 106. Alternatively, a single port device may be utilized such that the all agents are administered via the same port as with the exemplary syringe device of FIG. 2. The thymidine kinase gene may induce cell death in expressing cells in the presence of gancyclovir. Thus, TK-transfected cells may be sensitized to Gancyclovir. Therefore, breast cancer cells are killed after administration of Gancyclovir following PET scanning of the breast and detection of tumor.

The present invention provides a unique and novel method and apparatus for providing both diagnostic and therapeutic option in breast cancer management. By administering agents directly into the breast duct system, the tissue of interest receives the full administration of said agents and side effects are minimized. Previously, gancyclovir was administered systemically which exposed all organs to the compound. This possibly resulted in unwanted side effects that can be avoided in the method of the present invention. For example, systemic administration of Gancyclovir may result in anemia, neutropenia, thrombocytopenia, leukopenia, elevated creatinine or alkaline phosphatase, or elevated liver functions. Systemic Gancyclovir may also result in symptoms including abdominal pain, anorexia, fever, nausea, vomiting, diarrhea, headache, insommia, fatigue, dizziness, neurological symptoms, neuropathy, parathesias or rash, to name a few. Such signs and symptoms of side effects from gancyclovir may be avoided or minimized by local administration of the gancyclovir into the breast duct system rather than systemic administration. Moreover, local administration of gancyclovir, TK or acycloguanidines not only minimize side effects but also increase effectiveness of the agents because the compounds are concentrated at the site of interest, in this case, the breast. Thus, lower doses of the compound may be used as compared to systemic administration of the compounds thereby further reducing the risk of side effects.

Although the illustrative embodiments of the invention have been described, a wide range of modifications, changes and substitutions is intended in the foregoing disclosure. It is understood that the present invention can take many forms and embodiments. The embodiments shown herein are intended to illustrate rather than to limit the invention, it being appreciated that variations may be made without departing from the spirit of the scope of the invention.

What is claimed is:

1. A method for imaging breast lesions in a breast, the method comprising:
delivering a vector comprising a thymidine kinase gene into a breast duct of the breast;
administering an acycloguanidine compound into the breast duct; and
imaging the breast.

2. The method of claim 1 wherein the thymidine kinase gene is a herpes virus thymidine kinase gene.

3. The method of claim 1 wherein the vector and the acycloguanidine are administered into the breast duct simultaneously.

4. The method of claim 1 wherein the vector and the acycloguanidine are administered into the breast duct sequentially.

5. The method of claim 1 further comprising admininistering an acyclic nucleoside compound into the breast duct.

6. The method of claim 5 wherein the acyclic nucleoside compound is a gancyclovir.

7. The method of claim 1 wherein said imaging comprises PET scanning.

8. A method for imaging and treating breast lesions, the method comprising:
delivering a vector comprising a thymidine kinase gene into a breast duct of the breast;
administering an acycloguanidine compound into the breast duct;
administering to the breast duct an effective amount of an acyclic nucleoside compound wherein cancer cells are killed.

9. The method of claim 8 wherein the acyclic nucleoside compound is a gancyclovir.

10. The method of claim 8 wherein the thymidine kinase gene is a herpes virus thymidine kinase gene.

11. An apparatus for administering an agent into a breast duct comprising:
a catheter capable of being inserted into a breast duct;
a chamber containing a vector comprising a thymidine kinase gene.

12. The apparatus of claim 11 wherein the thymidine kinase gene is a herpes virus thymidine kinase gene.

13. The apparatus of claim 12 wherein the chamber further comprises an acycloguanidine compound.

14. The apparatus of claim 13 wherein the vector and the acycloguanidine compound are administered into the breast duct from the chamber simultaneously.

15. The apparatus of claim 11 further comprising a second chamber containing an acycloguanidine compound.

16. The apparatus of claim 15 wherein the vector and the acycloguanidine compound are administered into the breast duct simultaneously.

17. The apparatus of claim 15 wherein the vector and the acycloguanidine compound are administered into the breast duct sequentially.

18. An apparatus for administering an agent into a breast duct comprising:
a catheter capable of being inserted into a breast duct;
a chamber containing an acyclic nucleoside compound.

19. The apparatus of claim 18 wherein the acyclic nucleoside compound is a gancyclovir.

20. The apparatus of claim 18 wherein the acyclic nucleoside compound is administered into the breast duct.
